(19) United States
(12) Patent Application Publication
(10) Pub. No.: US 2022/0387640 A1
(43) Pub. Date: Dec. 8, 2022

(54) ULTRAVIOLET SANITIZING DEVICE

(71) Applicants: Andrew Winakor, Melville, NY (US); Michael Levine, Melville, NY (US); Dov Bloch, Ridgefield, CT (US)

(72) Inventors: Andrew Winakor, Melville, NY (US); Michael Levine, Melville, NY (US); Dov Bloch, Ridgefield, CT (US)

(21) Appl. No.: 17/342,408

(22) Filed: Jun. 8, 2021

(51) Int. Cl. A61L 2/10

(52) U.S. Cl. CPC A61L 2/10; A61L 2202/11; A61L 2202/14

(57) ABSTRACT

Apparatuses, systems, and methods are disclosed for an ultraviolet sanitation device. An apparatus includes an ultraviolet (“UV”) light emitter configured to emit UV light at a predefined wattage. The predefined wattage includes a wattage sufficient for ultraviolet germicidal irradiation. The apparatus includes a connector that corresponds to a connection port of a mobile device such that the UV light emitter is activated in response to the connector being connected to the connection port of the mobile device.

### FIELD

This invention relates to electronic devices and more particularly relates to an ultraviolet sanitizing device.

### BACKGROUND

Ultraviolet light can be used to sanitize and disinfect surfaces from harmful bacteria and other microorganisms.

### SUMMARY

Apparatuses, systems, and methods are disclosed for an ultraviolet sanitation device. An apparatus, in one embodiment, includes an ultraviolet (“UV”) light emitter configured to emit UV light at a predefined wattage. The predefined wattage includes a wattage sufficient for ultraviolet germicidal irradiation. The apparatus includes a connector that corresponds to a connection port of a mobile device such that the UV light emitter is activated in response to the connector being connected to the connection port of the mobile device.

An apparatus, in one embodiment, includes a gravity switch configured to activate the UV light emitter in response to being oriented at an angle that satisfies a predetermined angle relative to at least one of a horizontal plane and a vertical plane. In certain embodiments, an apparatus includes a controller configured to detect that the connector is connected to the connection port of the mobile device and activate the UV light emitter in response to detecting the connection.

In various embodiments, the controller is further configured to detect that the mobile device is one of turned off and placed in a sleep mode and deactivate the UV light emitter. In further embodiments, the controller is further configured to start a timer for a predefined amount of time in response to detecting the connection to the connection port of the mobile device and deactivate the UV light emitter in response to expiration of the timer.

In one embodiment, the predefined amount of time is within a range of one to five minutes. In some embodiments, the predefined amount of time is three minutes. In certain embodiments, an apparatus includes a plurality of UV light emitters, the UV light emitter being one of the plurality of UV light emitters.

In one embodiment, the connector is selected from the group comprising a lightning connector and a universal serial bus (“USB”) connector. In some embodiments, an apparatus includes a switch configured to activate and deactivate the UV light emitter. In various embodiments, an apparatus includes a control for adjusting a wattage of the UV light that the UV light emitter emits.

In one embodiment, an electronic device includes a housing and an apparatus located within the housing. In some embodiments, an apparatus includes an ultraviolet (“UV”) light emitter configured to emit UV light at a predefined wattage. The predefined wattage includes a wattage sufficient for ultraviolet germicidal irradiation. The apparatus includes a connector that corresponds to a connection port of a mobile device such that the UV light emitter is activated in response to the connector being connected to the connection port of the mobile device.

An apparatus, in one embodiment, includes a gravity switch configured to activate the UV light emitter in response to being oriented at an angle that satisfies a predetermined angle relative to at least one of a horizontal plane and a vertical plane.

In one embodiment, the housing comprises an opening that corresponds to the UV light emitter such that UV light that the UV light emitter emits is projected out of the housing via the opening. In certain embodiments, the housing comprises connection means for a keychain. In some embodiments, the connector is retractable into the housing via a retraction mechanism on the housing.

In one embodiment, a system includes a housing and an apparatus located within the housing. In some embodiments, an apparatus includes an ultraviolet (“UV”) light emitter configured to emit UV light at a predefined wattage. The predefined wattage includes a wattage sufficient for ultraviolet germicidal irradiation. The apparatus includes a connector that corresponds to a connection port of a mobile device such that the UV light emitter is activated in response to the connector being connected to the connection port of the mobile device.

An apparatus, in one embodiment, includes a gravity switch configured to activate the UV light emitter in response to being oriented at an angle that satisfies a predetermined angle relative to at least one of a horizontal plane and a vertical plane. In one embodiment, the system includes a plurality of different adaptors for the connector that each correspond to a different connection port type.

In one embodiment, the plurality of different adaptors comprises at least one of a lightning connection adaptor, a USB adaptor, a USB-C adaptor, and a micro-USB adaptor. In some embodiments, the system further includes at least one sensor for detecting a sanitation level of a surface where the UV light is emitted, wherein an alert is triggered in response to the detected sanitation level satisfying a threshold sanitation level.

In one embodiment, the system includes an application executing on the mobile device. The application may include code that is executable by a processor for managing the apparatus. In some embodiments, the executable code provides an interface for changing various settings of the apparatus. The various settings may include at least one of a wattage of the UV light, a length of time that the UV light emitter is active, selective control of each of a plurality of UV light emitters, activation/deactivation of the UV light emitter, and a sensitivity of the gravity switch.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1A depicts one embodiment of an ultraviolet sanitation device;

FIG. 1B depicts one embodiment of an opposite side of the ultraviolet sanitation device depicted in FIG. 1A;

FIG. 2 depicts another embodiment of an ultraviolet sanitation device;

FIG. 3 depicts a system for an ultraviolet sanitation device; and

FIG. 4 is a schematic flow chart diagram illustrating one embodiment of a method for an ultraviolet sanitation device.

### DETAILED DESCRIPTION

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments.

These features and advantages of the embodiments will become more fully apparent from the following description and appended claims or may be learned by the practice of embodiments as set forth hereinafter. As will be appreciated by one skilled in the art, aspects of the present invention may be embodied as a system, method, and/or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present invention may take the form of a computer program product embodied in one or more computer readable medium(s) having program code embodied thereon.

The described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

Many of the functional units described in this specification have been labeled as modules, in order to emphasize their implementation independence more particularly. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different storage devices, and may exist, at least partially, merely as electronic signals on a system or network. Where a module or portions of a module are implemented in software, the program code may be stored and/or propagated on in one or more computer readable medium(s).

The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (“RAM”), a read-only memory (“ROM”), an erasable programmable read-only memory (“EPROM” or Flash memory), a static random access memory (“SRAM”), a portable compact disc read-only memory (“CD-ROM”), a digital versatile disk (“DVD”), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (“ISA”) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (“LAN”) or a wide area network (“WAN”), or the connection may be made to an external computer (for example, through the Internet using an Internet Service Provider). In some embodiments, electronic circuitry including, for example, programmable logic circuitry, field-programmable gate arrays (“FPGA”), or programmable logic arrays (“PLA”) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The schematic flowchart diagrams and/or schematic block diagrams in the Figures illustrate the architecture, functionality, and operation of possible implementations of apparatuses, systems, methods, and computer program products according to various embodiments of the present invention. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

As used herein, a list with a conjunction of “and/or” includes any single item in the list or a combination of items in the list. For example, a list of A, B and/or C includes only A, only B, only C, a combination of A and B, a combination of B and C, a combination of A and C or a combination of A, B and C. As used herein, a list using the terminology “one or more of” includes any single item in the list or a combination of items in the list. For example, one or more of A, B and C includes only A, only B, only C, a combination of A and B, a combination of B and C, a combination of A and C or a combination of A, B and C. As used herein, a list using the terminology “one of” includes one and only one of any single item in the list. For example, “one of A, B and C” includes only A, only B or only C and excludes combinations of A, B and C. As used herein, “a member selected from the group consisting of A, B, and C,” includes one and only one of A, B, or C, and excludes combinations of A, B, and C.” As used herein, “a member selected from the group consisting of A, B, and C and combinations thereof” includes only A, only B, only C, a combination of A and B, a combination of B and C, a combination of A and C or a combination of A, B and C.

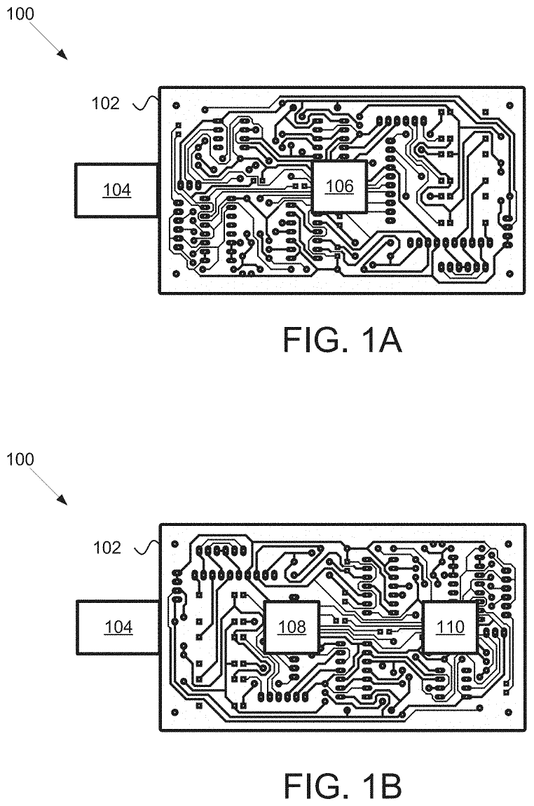

FIG. 1A depicts one embodiment of an ultraviolet sanitation device 100. In one embodiment, the ultraviolet sanitation device 100 comprises a printed circuit board (“PCB”) 102 comprising circuitry, chips, devices, and/or other hardware elements. FIG. 1A depicts one side of the PCB 102, where FIG. 1B depicts the opposite side of the PCB 102. In one embodiment, the PCB 102 includes a physical connector interface 104 for connecting to an external computing or electronic device such as a smart phone, a tablet computer, a laptop computer, a desktop computer, an external battery pack (e.g., a portable charger), and/or the like.

The interface 104, in one embodiment, may be embodied as a lightning connection, which may refer to a proprietary computer bus and power connector created and designed by Apple Inc. and is used to connect Apple mobile devices like iPhones, iPads, and iPods to host computers, external monitors, cameras, USB battery chargers, and other peripherals, e.g., the ultraviolet sanitizing device 100 described herein. In some embodiments, the interface 104 is embodied as a universal serial bus (“USB”) connector such as a USB-A, USB-B, USB-B mini, USB-B micro, USB-C, or the like for connecting to a device with a compatible or corresponding USB port. Other connection interfaces for connecting the ultraviolet sanitation device 100 may be envisioned in light of this disclosure.

In various embodiments, the ultraviolet sanitation device 100 includes different adaptors or connectors for the interface 104 that allows the interface 104 to connect to various connection types. For instance, if the interface 104 is a USB-C interface, but the user has a device with a lighting connection port, a lighting connection adapter may be attached to, slid on, put on, coupled to, or the like the interface 104 so that the ultraviolet sanitation device 100 is compatible with the lightning connection port.

In one embodiment, a hardware element connected to the PCB 102 may include an ultraviolet (“UV”) light emitter 106 that is configured to emit UV light at a predefined wattage. As used herein, UV light comprises a form of electromagnetic radiation with wavelength from 10 nm to 400 nm, shorter than that of visible light, but longer than X-rays. Forms of UV light includes ultraviolet C (“UV-C”), which has a wavelength in the range of 200 nm to 300 nm and ultraviolet A (“UV-A”), which has a wavelength in the range of 300 nm to 400 nm, both of which are useful for disinfection and sanitization— UV germicidal irradiation (“UVGI”). UVGI, as used herein, may refer to a disinfection method that uses ultraviolet (e.g., UV-C) light to kill or inactivate microorganisms, e.g., bacteria and viruses. In one embodiment, the UV light that is emitted may include UV-C light, e.g., within a range of 200-280 nm, including short-wavelength UV light that is around 254 nm and/or far-wavelength UV light that is around 222 nm.

The UV light emitter 106 may be embodied as a UV light emitting diode (“LED”), or similar component. Even though a single UV light emitter 106 is depicted in FIG. 1A, the ultraviolet sanitation device 100 may include a plurality of UV light emitters 106, e.g., an array of UV light emitters 106.

In one embodiment, shown in FIG. 1B, the ultraviolet sanitation device 100 may include a gravity switch 108. In some embodiments, the UV light emitter 106 and the gravity switch 108 are located on the same side of the PCB 102, on opposite sides of the PCB 102 (as shown in FIGS. 1A and 1B, which helps with actuation when the UV light emitter 106 is oriented upside down, as explained below), at various locations on a side of the PCB 102, or the like.

The gravity switch 108, as used herein, comprises an electrical switch that is actuated by gravity. In one embodiment, the gravity switch 108 is actuated to activate/deactivate or turn on/off the UV light emitter 106. In such an embodiment when the gravity switch 108 is oriented at a particular angle, e.g., 45° or more relative to a horizontal or vertical plane, the gravity switch is actuated to deactivate or turn the UV light emitter 106 off. In this manner, the gravity switch 108 serves a safety function by inhibiting or preventing the functioning of the UV light emitter 106 when it is elevated or angled beyond a certain vertical point, relative to a horizontal plane, and/or is used upside down to prevent inadvertent light exposure to humans, pets, plants, and/or the like.

In one embodiment, the ultraviolet sanitation device 100 is configured, e.g., electrically designed, or hardwired, to activate the UV light emitter 106 when power is provided to the ultraviolet sanitation device 100 via the interface 104. For example, when the interface 104 for the ultraviolet sanitation device 100 is connected to an iPhone®, and power is provided from the iPhone® to the ultraviolet sanitation device 100, the UV light emitter 106 is activated. In certain embodiments, however, if the gravity switch 106 is oriented at an angle greater than a predefined or set threshold angle relative to a horizontal plane, the gravity switch 106 may be actuated to deactivate, turn off, or cut power to the UV light emitter 106 for safety.

In one embodiment, the ultraviolet sanitation device 100 includes a controller 110 that is configured to control, manage, direct, or the like various functions or operations of the ultraviolet sanitation device 100. The controller 110 may be embodied as a specially-programmed hardware device or integrated circuit, e.g., a field programmable gate array, an application specific integrated circuit, or the like, and may include firmware or other software for controlling the ultraviolet sanitation device 100.

In one embodiment, the controller 110 is configured to detect that the connector interface 104 is connected to a connection port of the mobile or electronic device and activate the UV light emitter 106 in response to detecting the connection. In such an embodiment, the controller 110 may detect power or other signal in response to the interface 104 being connected to a device and may cause, trigger, signal, or the like the UV light emitter 106 to be activated or turned on (if the gravity switch is oriented such that it allows the UV light emitter 106 to be enabled).

In one embodiment, the controller 110 is further configured to detect that the electronic or mobile device is turned off, placed in a sleep mode, and/or the like and deactivate the UV light emitter 106. In such an embodiment, the controller 110 may receive a signal or other notification, e.g., from the device, that indicates that the electronic device that the ultraviolet sanitation device 100 is connected to has been deactivated, placed in sleep mode, turned off, or the like, and may cause, signal, trigger, or the like the UV light emitter 106 to be deactivated or turned off.

In one embodiment, the controller 110 is further configured to start a timer for a predefined amount of time in response to detecting that the ultraviolet sanitation device 100 is connected to the connection port of the electronic or mobile device and the UV light emitter 106 is activated. For instance, the controller 110 may start a timer for 15 seconds, 30 seconds, one minute, three minutes, five minutes, and/or the like. On expiration of the timer, the controller 110 may deactivate the UV light emitter 106. In this manner, the UV light emitter 106 is automatically disabled or turned off after a period of time, even if it is still connected to the electronic or mobile device, which maintains the life of the UV light emitter 106 and also prevents inadvertent exposure to the UV-C light if the user, for example, forgets that the ultraviolet sanitation device 100 is connected to the electronic or mobile device.

In one embodiment, the ultraviolet sanitation device 100 may include a physical or manual switch, button, or the like (not shown), that a user can actuate to manually turn the UV light emitter on and off. In such an embodiment, the gravity switch 108 may still be active to allow or prevent activation of the UV light emitter 106 dependent on the orientation and/or angle of the gravity switch 108, as described above. Furthermore, the ultraviolet sanitation device 100 may include a physical control (not shown) such as a spin control, up/down buttons, and/or the like, for adjusting the wattage, power, or brightness of the UV light that the UV light emitter 106 emits.

In one embodiment, the ultraviolet sanitation device 100 may include one or more sensors (not shown) for detecting a sanitation level of a surface where the UV light emitter 106 is emitted. In such an embodiment, the sensors may include biosensors, temperature sensors, or the like for detecting different levels of environmental characteristics that are indicative of a sanitary surface. The sensors may be activated in response to the UV light emitter 106 being activated, in response to the ultraviolet sanitation device 100 being connected to and powered by a device, and/or the like. In various embodiments, the sensors may provide feedback on the sanitation level, e.g., a different signal voltage indicating whether the surface is sanitary or not, that the controller 110 can use to provide feedback (e.g., such as a beep or other sound/signal) to indicate whether the surface is sanitary or not according to a predefined threshold level.

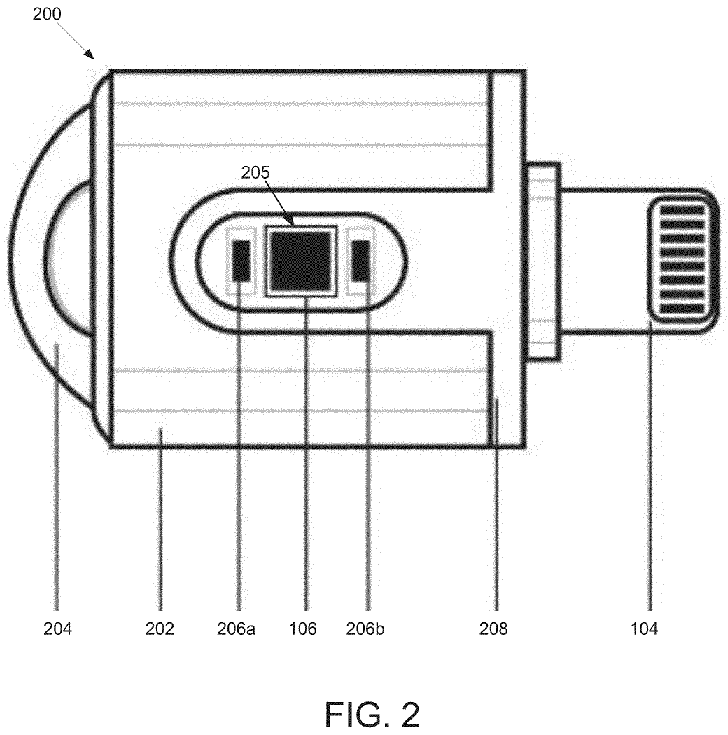

FIG. 2A depicts one embodiment of an ultraviolet sanitation device 100 that is embedded, encompassed, inserted, or otherwise housed within a housing 200. The housing 200 may include an external shell 202 that is made of plastic, aluminum, steel, and/or the like. The ultraviolet sanitation device 100 may be inserted into the external shell 202. In certain embodiments, the ultraviolet sanitation device 100 is embedded, encompassed, inserted, or otherwise housed within an inner shell middle bracket 208, which is then inserted into the external shell 202. This may allow for the interchangeability of different external shells 202. The inner shell middle bracket 208 may be made of plastic, aluminum, steel, and/or the like.

In one embodiment, the housing 200 includes a connection member 204 for attaching the housing 200 to a hook, keychain, or the like. In certain embodiments, the housing 200 includes an opening or window 205 for exposing the UV light emitter 106 so that UV light, e.g., UV-C light can be emitted from the ultraviolet sanitation device 100 for sanitizing objects within the direction of the UV light emitter 106. In certain embodiments, the housing 200 may include a slider or shutter (not shown) that is used to close or cover the window to prevent UV light from being inadvertently emitted from UV light emitter 106 of the ultraviolet sanitation device 100.

In one embodiment, the housing 200 may include controls 206a-b such as buttons, switches, spin control, or the like for controlling different features of the ultraviolet sanitation device 100, such as turning the UV light emitter 106 on/off, adjusting the wattage of the UV light emitter 106, and/or the like. In further embodiments, the housing 200 may include a slider or other retraction mechanism for retracting the interface 104 into the housing 200. In certain embodiments, the housing 200 may include a corresponding cap or cover (not shown) that is configured to cover the interface 104. Such a cap or cover may be tethered to the housing 200, e.g., to the external shell 202, or may be a completely separate piece.

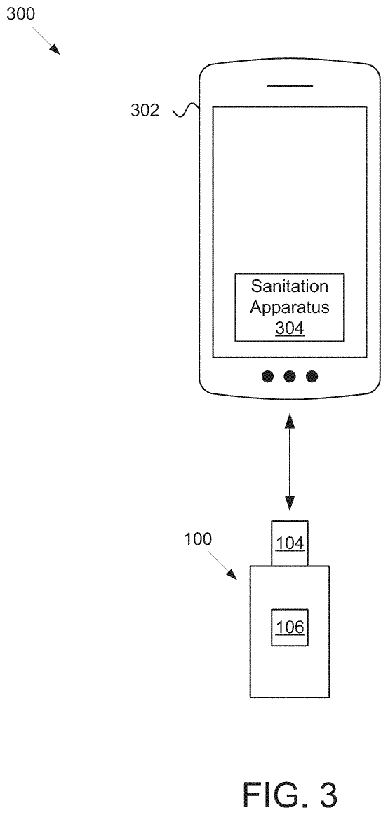

FIG. 3 depicts one embodiment of a system 300 for an ultraviolet sanitation device 100. In one embodiment, the system 300 includes an ultraviolet sanitation device 100, which may be housed in a housing 200 as depicted in FIG. 2. Furthermore, the system 300 includes an electronic or mobile device 302 such as a mobile phone, a tablet computer, a smart watch, a laptop computer, a desktop computer, and/or the like.

In one embodiment, the electronic or mobile device 302 includes a sanitation apparatus 304. The sanitation apparatus 304, in one embodiment, is configured to communicate with the ultraviolet sanitation device 100 to control, manage, configure, or the like the ultraviolet sanitation device 100. For instance, the sanitation apparatus 304 may comprise an application that is executable on the mobile device 302 that is programmed or configured for controlling various settings of the ultraviolet sanitation device 100 such as turning the UV light emitter 106 on/off, controlling a wattage, power, or brightness of the UV light emitter 106, tracking how long the UV light emitter 106 has been used, estimating or forecasting a remaining lifetime of the UV light emitter 106, setting thresholds such as the sanitation level threshold, updating firmware for various hardware components of the ultraviolet sanitation device 100, and/or the like.

In such an embodiment, the sanitation apparatus 304 provides a graphical interface, a web interface, or the like for setting, modifying, adjusting, changing, or the like configuration settings or parameters for the ultraviolet sanitation device 100. For example, the interface may allow a user to interact with graphical control elements for turning the UV light emitter 106 on/off, for setting (e.g., increasing or decreasing) the wattage used for powering the UV light emitter 106, for setting threshold values, for selecting one or more subsets of UV light emitters 106 to be activated, setting a sensitivity level for the gravity switch, and/or the like.

In one embodiment, the sanitation apparatus 304 may be in communication with sensors on the ultraviolet sanitation device 100 to detect, monitor, and/or report the sanitation level of a surface; to provide information regarding a distance between the ultraviolet sanitation device 100 and the surface and provide recommendations for moving the ultraviolet sanitation device 100 closer or further away; and/or the like. The sanitation apparatus 304 may further provide information on an interface regarding a timer or clock to countdown or inform the user as to how long to expose a particular surface to the UV light; provide readings from the gravity switch, e.g., information about the orientation angle that the gravity switch is currently at and at what angle it is activated; show usage data such as how long the UV light emitter 106 has been used, how much estimated lifetime is remaining, and/or the like; provide current wattage or power information and recommendations for increasing or decreasing the wattage for a particular surface; and/or the like.

In certain embodiments, the sanitation apparatus 304 may include a hardware device such as a secure hardware dongle or other hardware appliance device (e.g., a set-top box, a network appliance, or the like) that attaches to a device such as a head mounted display, a laptop computer, a server 108, a tablet computer, a smart phone, a security system, a network router or switch, or the like, either by a wired connection (e.g., a universal serial bus (“USB”) connection) or a wireless connection (e.g., Bluetooth®, Wi-Fi, near-field communication (“NFC”), or the like); that attaches to an electronic display device (e.g., a television or monitor using an HDMI port, a DisplayPort port, a Mini DisplayPort port, VGA port, DVI port, or the like); and/or the like. A hardware appliance of the sanitation apparatus 304 may include a power interface, a wired and/or wireless network interface, a graphical interface that attaches to a display, and/or a semiconductor integrated circuit device as described below, configured to perform the functions described herein with regard to the sanitation apparatus 304.

The sanitation apparatus 304, in such an embodiment, may include a semiconductor integrated circuit device (e.g., one or more chips, die, or other discrete logic hardware), or the like, such as a field-programmable gate array (“FPGA”) or other programmable logic, firmware for an FPGA or other programmable logic, microcode for execution on a microcontroller, an application-specific integrated circuit (“ASIC”), a processor, a processor core, or the like. In one embodiment, the sanitation apparatus 304 may be mounted on a printed circuit board with one or more electrical lines or connections (e.g., to volatile memory, a non-volatile storage medium, a network interface, a peripheral device, a graphical/display interface, or the like). The hardware appliance may include one or more pins, pads, or other electrical connections configured to send and receive data (e.g., in communication with one or more electrical lines of a printed circuit board or the like), and one or more hardware circuits and/or other electrical circuits configured to perform various functions of the sanitation apparatus 304.

The semiconductor integrated circuit device or other hardware appliance of the sanitation apparatus 304, in certain embodiments, includes and/or is communicatively coupled to one or more volatile memory media, which may include but is not limited to random access memory (“RAM”), dynamic RAM (“DRAM”), cache, or the like. In one embodiment, the semiconductor integrated circuit device or other hardware appliance of the sanitation apparatus 304 includes and/or is communicatively coupled to one or more non-volatile memory media, which may include but is not limited to: NAND flash memory, NOR flash memory, nano random access memory (nano RAM or “NRAM”), nanocrystal wire-based memory, silicon-oxide based sub-10 nanometer process memory, graphene memory, Silicon-Oxide-Nitride-Oxide-Silicon (“SONOS”), resistive RAM (“RRAM”), programmable metallization cell (“PMC”), conductive-bridging RAM (“CBRAM”), magneto-resistive RAM (“MRAM”), dynamic RAM (“DRAM”), phase change RAM (“PRAM” or “PCM”), magnetic storage media (e.g., hard disk, tape), optical storage media, or the like.

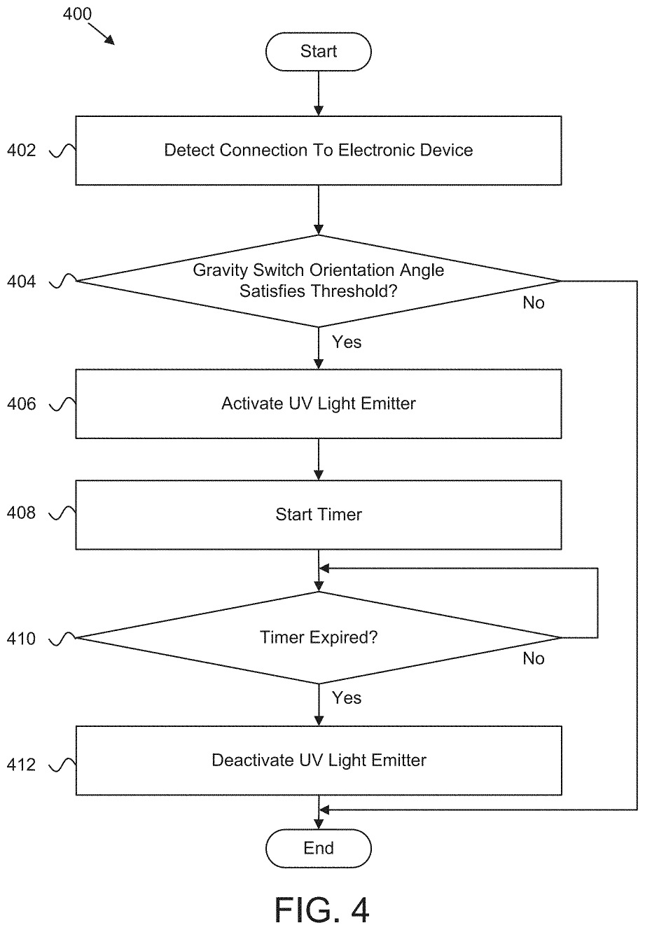

FIG. 4 depicts a schematic flow chart diagram illustrating one embodiment of a method for an ultraviolet sanitation device 100. In one embodiment, the method 400 begins and detects 402 a connection of the ultraviolet sanitation device 100 to an electronic device. The method 400, in further embodiments, determines 404 whether a gravity switch of the ultraviolet sanitation device 100 is at an orientation angle that satisfies a threshold orientation angle, e.g., 45° or less. If not, the method 400 ends.

Otherwise, in one embodiment, the method 400 activates 406, e.g., turns on, a UV light emitter 106 of the ultraviolet sanitation device 100. The method 400, in further embodiments, starts 408 a timer that is set for a duration that the UV light emitter 106 is activated. In one embodiment, the method 400 determines 410 whether the timer has expired. If not, the method 400 continues to determine 410 whether the timer has expired. Otherwise, in one embodiment, if the timer has expired, the method 400 deactivates the UV light emitter 106, and the method 400 ends.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus, comprising:
an ultraviolet (“UV”) light emitter configured to emit UV light at a predefined wattage, the predefined wattage comprising a wattage for ultraviolet germicidal irradiation; and
a connector that corresponds to a connection port of a mobile device such that the UV light emitter is activated in response to the connector being connected to the connection port of the mobile device.

2. The apparatus of claim 1, further comprising a gravity switch configured to activate the UV light emitter in response to being oriented at an angle that satisfies a predetermined angle relative to at least one of a horizontal plane and a vertical plane.

3. The apparatus of claim 1, further comprising a controller configured to detect that the connector is connected to the connection port of the mobile device and activate the UV light emitter in response to detecting the connection.

4. The apparatus of claim 3, wherein the controller is further configured to detect that the mobile device is one of turned off and placed in a sleep mode and deactivate the UV light emitter.

5. The apparatus of claim 3, wherein the controller is further configured to start a timer for a predefined amount of time in response to detecting the connection to the connection port of the mobile device and deactivate the UV light emitter in response to expiration of the timer.

6. The apparatus of claim 5, wherein the predefined amount of time is within a range of one to five minutes.

7. The apparatus of claim 6, wherein the predefined amount of time is three minutes.

8. The apparatus of claim 1, further comprising a plurality of UV light emitters, the UV light emitter being one of the plurality of UV light emitters.

9. The apparatus of claim 1, wherein the connector is selected from the group comprising a lightning connector and a universal serial bus (“USB”) connector.

10. The apparatus of claim 1, further comprising a switch configured to activate and deactivate the UV light emitter.

11. The apparatus of claim 1, further comprising a control for adjusting a wattage of the UV light that the UV light emitter emits.

12. An electronic device comprising:
a housing; and
an apparatus located within the housing, the apparatus comprising:
an ultraviolet (“UV”) light emitter configured to emit UV light at a predefined wattage, the predefined wattage comprising a wattage for ultraviolet germicidal irradiation;
a connector that corresponds to a connection port of a mobile device such that the UV light emitter is activated in response to the connector being connected to the connection port of the mobile device; and
a gravity switch configured to activate the UV light emitter in response to being oriented at an angle that satisfies a predetermined angle relative to at least one of a horizontal plane and a vertical plane.

13. The electronic device of claim 12, wherein the housing comprises an opening that corresponds to the UV light emitter such that UV light that the UV light emitter emits is projected out of the housing via the opening.

14. The electronic device of claim 12, wherein the housing comprises connection means for a keychain.

15. The electronic device of claim 12, wherein the connector is retractable into the housing via a retraction mechanism on the housing.

16. A system, comprising:
a housing;
an apparatus located within the housing, the apparatus comprising:
an ultraviolet (“UV”) light emitter configured to emit UV light at a predefined wattage, the predefined wattage comprising a wattage for ultraviolet germicidal irradiation;
a connector that corresponds to a connection port of a mobile device such that the UV light emitter is activated in response to the connector being connected to the connection port of the mobile device; and
a gravity switch configured to activate the UV light emitter in response to being oriented at an angle that satisfies a predetermined angle relative to at least one of a horizontal plane and a vertical plane; and
a plurality of different adaptors for the connector that each correspond to a different connection port type.

17. The system of claim 16, wherein the plurality of different adaptors comprises at least one of a lightning connection adaptor, a USB adaptor, a USB-C adaptor, and a micro-USB adaptor.

18. The system of claim 16, further comprising at least one sensor for detecting a sanitation level of a surface where the UV light is emitted, wherein an alert is triggered in response to the detected sanitation level satisfying a threshold sanitation level.

19. The system of claim 16, further comprising an application executing on the mobile device, the application comprising code executable by a processor for managing the apparatus.

20. The system of claim 19, wherein the executable code provides an interface for changing various settings of the apparatus, the various settings comprising at least one of a wattage of the UV light, a length of time that the UV light emitter is active, selective control of each of a plurality of UV light emitters, activation/deactivation of the UV light emitter, and a sensitivity of the gravity switch.
